(19) United States
(12) Patent Application Publication
(10) Pub. No.: US 2024/0269138 A1
(43) Pub. Date: Aug. 15, 2024

(54) MMP13 AS A THERAPEUTIC TARGET FOR ALLERGIC INFLAMMATORY DISEASES

(71) Applicant: The General Hospital Corporation, Boston, MA (US)

(72) Inventors: Jin Mo Park, Newton, MA (US); Yanek Jimenez-Andrade, Brighton, MA (US)

(21) Appl. No.: 18/563,575

(22) PCT Filed: May 24, 2022
(86) PCT No.: PCT/US22/72524

§ 371 Date: Nov. 22, 2023

Related U.S. Application Data
(60) Provisional application No. 63/192,867, filed on May 25, 2021.

(51) Int. Cl. A61K 31/505; A61K 31/5377; A61K 45/06; A61P 37/08

(52) U.S. Cl. CPC A61K 31/505; A61K 31/5377; A61K 45/06; A61P 37/08

(57) ABSTRACT

Aspects of the present disclosure provide methods for treating allergic inflammatory diseases, such as allergic asthma and atopic dermatitis, using inhibitors of matrix metalloproteinase 13 (MMP13) such as small molecule inhibitors of MMP13.

### CLAIM OF PRIORITY

This application claims the benefit of U.S. Provisional Patent Application No. 63/192,867, filed on May 25, 2021, which is incorporated by reference herein in its entirety.

### FEDERALLY SPONSORED RESEARCH OR DEVELOPMENT

This invention was made with government support under Grant No. AI127768 awarded by the National Institutes of Health. The government has certain rights in the invention.

### FIELD OF THE INVENTION

The subject matter disclosed herein generally relates to methods for treating allergic inflammatory diseases.

### BACKGROUND OF THE INVENTION

Allergic inflammatory diseases such as atopic dermatitis and asthma are major health threats worldwide, and collectively pose an enormous socioeconomic burden with their high prevalence and growing incidence. Anti-inflammatory steroid agents and biologics are available to treat atopic dermatitis and asthma. These currently available treatment options, however, offer limited effectiveness due to widely varying efficacy, adverse side effects, treatment resistance, and prohibitively high costs. Therefore, there is a great need for new therapies that are more broadly effective against allergic diseases.

### SUMMARY OF THE INVENTION

The present disclosure is based, at least in part, on the identification of matrix metalloproteinase 13 (MMP13) as an epithelial-derived inflammatory mediator whose expression depends on signaling by the stress- and inflammation-activated protein kinase p38α. Experimental data provided herein demonstrated that systemic or epithelial-restricted deletion of the MMP13 gene attenuated allergic inflammation and prevented tissue pathology in mouse models of atopic dermatitis and asthma. It was also demonstrated that a topically applied MMP13 inhibitor was effective at suppressing allergic dermatitis in mice.

Accordingly, aspects of the present disclosure provide a method for treating an allergic inflammatory disease comprising administering to a subject in need thereof an effective amount of an inhibitor of matrix metalloproteinase 13 (MMP13).

In some embodiments, the inhibitor of MMP13 is selected from the group consisting of a small molecule inhibitor, an anti-MMP13 antibody, a peptide inhibitor, and an agent that inhibits expression of MMP13. In some embodiments, the inhibitor of MMP13 is a small molecule inhibitor of MMP13. In some embodiments, the small molecule inhibitor is a non-Zn²⁺-binding MMP13 inhibitor. In some embodiments, the small molecule inhibitor is selected from the group consisting of CL82198 (CAS 307002-71-7), DB04760 (CAS 544678-85-5), WAY 170523 (CAS 307002-73-9), MMP-9/MMP-13 inhibitor I (CAS 204140-01-2), PD166793 (CAS 199850-67-4), and BI-4394 (CAS 1222173-37-6). In some embodiments, the small molecule inhibitor is CL82198 (CAS 307002-71-7). In some embodiments, the small molecule inhibitor is DB04760 (CAS 544678-85-5).

In some embodiments, the inhibitor of MMP13 is formulated for topical administration. In some embodiments, the inhibitor of MMP13 is formulated for inhalation. In some embodiments, the inhibitor of MMP13 is formulated in a pharmaceutical composition, which further comprises a pharmaceutically acceptable carrier.

In some embodiments, the subject is a human patient having or at risk for having an allergic inflammatory disease. In some embodiments, the allergic inflammatory disease is selected from the group consisting of allergic asthma, atopic dermatitis, allergic rhinitis, and allergic conjunctivitis. In some embodiments, the allergic inflammatory disease is allergic asthma. In some embodiments, the allergic inflammatory disease is atopic dermatitis.

In some embodiments, the inhibitor of MMP13 is administered topically. In some embodiments, the inhibitor of MMP13 is administered systemically. In some embodiments, the inhibitor of MMP13 is administered by inhalation.

In some embodiments, methods described herein further comprise administering to the subject an additional therapeutic agent. In some embodiments, the additional therapeutic agent is selected from the group consisting of an anti-inflammatory agent, an antihistamine, an immunosuppressant, an IL-4Rα neutralizing agent, and a JAK inhibitor.

In some embodiments, the inhibitor of MMP13 and the additional therapeutic agent are formulated in a single formulation.

### BRIEF DESCRIPTION OF THE DRAWINGS

FIGS. 1A-1G include data showing that epithelial-restricted deletion of the p38α gene suppresses inflammation in a mouse model of atopic dermatitis. WT and p38α-EKO mice were sensitized to the experimental protein allergen ovalbumin (OVA) by intraperitoneal injection on day 0 and 7. The sensitized mice were challenged epicutaneously with control saline (SAL) or OVA (n=7 per group) on days 14, 16 and 19. SAL- and OVA-challenged skin was prepared on day 21 for analysis. (FIGS. 1A-1C) Skin sections were analyzed by H&E staining (FIG. 1A). Epidermal and dermal thickness (FIG. 1B and FIG. 1C, respectively) was determined from H&E-stained skin images and are shown as means SEM. *, P<0.05; **, P<0.01 (two-tailed unpaired Student's t test). (FIGS. 1D-1G) Skin sections were analyzed by immunostaining/counterstaining for the indicated molecules (FIG. 1D and FIG. 1F). Solid and dotted line, epidermal margins and the epidermal-dermal boundary, respectively. CD3⁺ T cell and MBP⁺ eosinophil density (FIG. 1E and FIG. 1G, respectively) was determined on the basis of fluorescence signal-bearing cell numbers per image field and is shown as means±SEM. *, P<0.05; **, P<0.01 (two-tailed unpaired Student's t test).

FIGS. 2A-2I include data showing that deletion of the MMP13 gene suppresses inflammation in a mouse model of atopic dermatitis. (FIG. 2A and FIG. 2B) Parental WT and CRISPR-engineered p38α-KO HaCaT keratinocytes were left unstimulated or stimulated with IL-1α (20 ng/ml). RNA was prepared 4 hours later and analyzed by RNA-seq (FIG. 2A) and qPCR (FIG. 2B). Relative mRNA amounts for individual genes are presented on a color-coded scale shown at the bottom. C1 and C2, independent CRISPR-engineered clones. (FIGS. 2C-2I) WT and MMP13-KO mice were sensitized to OVA and challenged with control SAL or OVA (n=9 and 6 per group, respectively) as in FIGS. 1A-1G. SAL- and OVA-challenged skin was prepared for analysis as in FIGS. 1A-1G. Skin sections were analyzed by H&E staining (FIG. 2C). Epidermal and dermal thickness (FIG. 2D and FIG. 2E, respectively) was determined from H&E-stained skin images and are shown as means±SEM. *, P<0.05; ****, P<0.0001 (two-tailed unpaired Student's t test). Skin sections were analyzed by immunostaining/counterstaining for the indicated molecules (FIG. 2F and FIG. 2H). Solid and dotted line, epidermal margins and the epidermal-dermal boundary, respectively. CD3⁺ T cell and MBP⁺ eosinophil density (FIG. 2G and FIG. 2I, respectively) was determined on the basis of fluorescence signal-bearing cell numbers per image field and is shown as means±SEM. *, P<0.05 (two-tailed unpaired Student's t test).

FIGS. 3A-3K include data showing that epithelial-restricted deletion of the MMP13 gene suppresses inflammation in a mouse model of atopic dermatitis. (FIG. 3A and FIG. 3B) Skin sections from mice with OVA-induced allergic dermatitis (FIG. 3A) and TPA-induced acute dermatitis (FIG. 3B) were analyzed by immunostaining/counterstaining for the indicated molecules. Dotted line, the epidermal-dermal boundary. (FIG. 3C and FIG. 3D) Primary human keratinocytes were left unstimulated (None) or stimulated with the indicated cytokines (20 ng/ml). RNA was prepared 4 hours later and analyzed by qPCR. Relative mRNA amounts for individual genes are presented on a color-coded scale shown at the bottom. (FIGS. 3E-3K) WT and MMP13-EKO mice were sensitized to OVA and challenged with control SAL or OVA (n=8 per group) as in FIGS. 1A-1G. SAL- and OVA-challenged skin was prepared for analysis as in FIGS. 1A-1G. Skin sections were analyzed by H&E staining (FIG. 3E). Epidermal and dermal thickness (FIG. 3F and FIG. 3G, respectively) was determined from H&E-stained skin images and are shown as means±SEM. *, P<0.05 (two-tailed unpaired Student's t test). Skin sections were analyzed by immunostaining/counterstaining for the indicated molecules (FIG. 3H and FIG. 3J). Solid and dotted line, epidermal margins and the epidermal-dermal boundary, respectively. CD3⁺ T cell and MBP⁺ eosinophil density (FIG. 3I and FIG. 3K, respectively) was determined on the basis of fluorescence signal-bearing cell numbers per image field and is shown as means±SEM. *, P<0.05 (two-tailed unpaired Student's t test).

FIGS. 4A-4G include data showing that topical administration of a small-molecule inhibitor of p38α or MMP13 suppresses inflammation in a mouse model of atopic dermatitis. C57BL/6 mice were sensitized to OVA and challenged with control SAL or OVA as in FIGS. 1A-1G. Vehicle (water), the p38α inhibitor SCIO469 (p38αi), and the MMP13 inhibitor DB04760 (CAS 544678-85-5; MMP13i) were administered topically to the challenged skin site (125 μl of 50 mM solution per cm²; n=10 per group) concurrently with SAL or OVA. SAL- and OVA-challenged skin was prepared for analysis as in FIGS. 1A-1G. (FIGS. 4A-4C) Skin sections were analyzed by H&E staining (FIG. 4A). Epidermal and dermal thickness (FIG. 4B and FIG. 4C, respectively) was determined from H&E-stained skin images and are shown as means±SEM. *, P<0.05; **, P<0.01 (two-tailed unpaired Student's t test). (FIGS. 4D-4G) Skin sections were analyzed by immunostaining/counterstaining for the indicated molecules (FIG. 4D and FIG. 4F). Solid and dotted line, epidermal margins and the epidermal-dermal boundary, respectively. CD3⁺ T cell and MBP⁺ eosinophil density (FIG. 4E and FIG. 4G, respectively) was determined on the basis of fluorescence signal-bearing cell numbers per image field and is shown as means SEM. **, P<0.01; ***, P<0.001 (two-tailed unpaired Student's t test).

FIGS. 5A-5D include data showing that deletion of the MMP13 gene suppresses inflammation in a mouse model of asthma WT and MMP13-KO mice were subjected to intranasal instillation of control PBS or the model allergen HDM (n=11 and 10 per group, respectively) on days 0, 2, 5, 7, 9, 12, 14, 16 and 19. BAL and lungs of PBS- and HDM-challenged mice were prepared on day 21 for analysis. (FIG. 5A and FIG. 5B) BAL cells were analyzed by Diff-Quik staining (FIG. 5A). The numbers of total BAL cells and the indicated types of BAL cells were determined from Diff-Quik-stained cell images and are shown as means±SEM (FIG. 5B). *, P<0.05; ***, P<0.001 (two-tailed unpaired Student's t test). (FIG. 5C and FIG. 5D) Lung sections were analyzed by H&E staining (FIG. 5C) and PAS staining (FIG. 5D).

FIGS. 6A-6D include data showing that systemic administration of small-molecule MMP13 inhibitors suppresses inflammation in a mouse model of asthma. C57BL/6 mice were subjected to intranasal instillation of control PBS or HDM as in FIGS. 5A-5D. Vehicle (DMSO), dexamethasone (1 mg/kg), and the MMP13 inhibitors CL82198 (MMP13i Inh1; 50 mg/kg) and DB04760 (CAS 544678-85-5; MMP13i Inh2; 50 mg/kg) were administered intraperitoneally (n=3, 3, 4, and 2 per group, respectively) on days 19 and 20. BAL and lungs of PBS- and HDM-challenged mice were prepared for analysis as in FIGS. 5A-5D. (FIG. 6A and FIG. 6B) BAL cells were analyzed by Diff-Quik staining (FIG. 6A). The numbers of total BAL cells and the indicated types of BAL cells were determined from Diff-Quik-stained cell images and are shown as means±SEM (FIG. 6B). *, P<0.05 (two-tailed unpaired Student's t test). (FIG. 6C and FIG. 6D) Lung sections were analyzed by H&E staining (FIG. 6C) and PAS staining (FIG. 6D).

FIG. 7 includes structures of two small-molecule inhibitors, CL82198 and DB04760 (CAS 544678-85-5), which were used in studies described herein.

The details of one or more embodiments of the invention are set forth in the description below. Other features or advantages of the present invention will be apparent from the following drawings and detailed description of several embodiments, and also from the appended claims.

### DETAILED DESCRIPTION

The present disclosure is based, at least in part, on the finding that genetic ablation of matrix metalloproteinase 13 (MMPP13) or treatment with small molecule inhibitors of MMP13 suppressed inflammatory responses and tissue alteration in atopic dermatitis and asthma. It was also demonstrated that MMP13, whose expression in epithelial cells depends on p38α, functions as a novel molecular mediator linking p38α signaling to allergic inflammation.

Allergic immune responses occur most prominently in skin and mucosal surfaces—where defense against macroparasites and environmental toxins is most desperately needed and can become most effective—and are mediated by specific cellular and molecular effectors of immunity such as eosinophils, immunoglobulin (Ig) E, and mucus secretion. In addition, itch-induced scratching and respiratory/gastrointestinal smooth muscle contraction serve as additional mechanisms for the detachment or expulsion of parasites and toxins. When excessive in its intensity and duration and if misdirected against innocuous environmental agents, allergic responses cause tissue damage, organ dysfunction, and other pathologic conditions of varying seriousness, ranging from a minor annoyance to life-threatening disease.

A multitude of cell types, parenchymal or hematopoietic in origin, participate in the pathogenesis of allergic disease. Dendritic cells present in barrier tissues capture allergens, migrate to the draining lymph node, and presenting them to naïve T cells. Allergen-primed T cells acquire specific T helper (Th) functional properties and are recruited to allergen-exposed tissue sites. Pivotal to mobilizing allergic effector mechanisms is the function of Th2 cell-derived cytokines (e.g., interleukin [IL]-4, IL-5, IL-13). Th17 cell-derived cytokines (e.g., IL-17A, IL-22) also contribute to allergic disease in certain types of patients. Barrier tissue epithelial cells such as epidermal keratinocytes and airway epithelial cells produce cytokines, chemokines, and other mediators of allergic immune responses, thereby promoting or regulating the trafficking and function of dendritic cells, T cells, and other hematopoietic-derived cells. The current understanding of how barrier epithelia contribute to allergic disease, however, remains rudimentary and has not translated into highly effective therapies.

The protein kinase p38α is expressed in most mammalian cell types, including keratinocytes and airway epithelial cells, and activated by pro-inflammatory cytokines, microbial immunostimulatory products, and injurious environmental insults. A multitude of p38α inhibitors were developed and tested in clinical studies evaluating their efficacy for rheumatoid arthritis, chronic obstructive pulmonary disease, Crohn's disease, and other indications (Arthur & Ley, Mitogen-activated protein kinases in innate immunity. Nat Rev Immunol. 2013, 13, 679-692). In these studies, p38α inhibition showed some efficacy but produced hepatic, cutaneous and gastrointestinal toxicity (Salgado et al., Safety profile of protein kinase inhibitors in rheumatoid arthritis: systematic review and meta-analysis. Ann Rheum Dis 2014, 73, 871-882). These adverse effects were thought to arise from interference with beneficial homeostatic functions of p38α. These results illustrated a need for improved strategies for blocking p38α-driven inflammatory responses. Conceivable strategies include inhibiting molecular events downstream of p38α or targeting specific cell types in which p38α signaling serves functions linked to disease but not essential homeostatic processes.

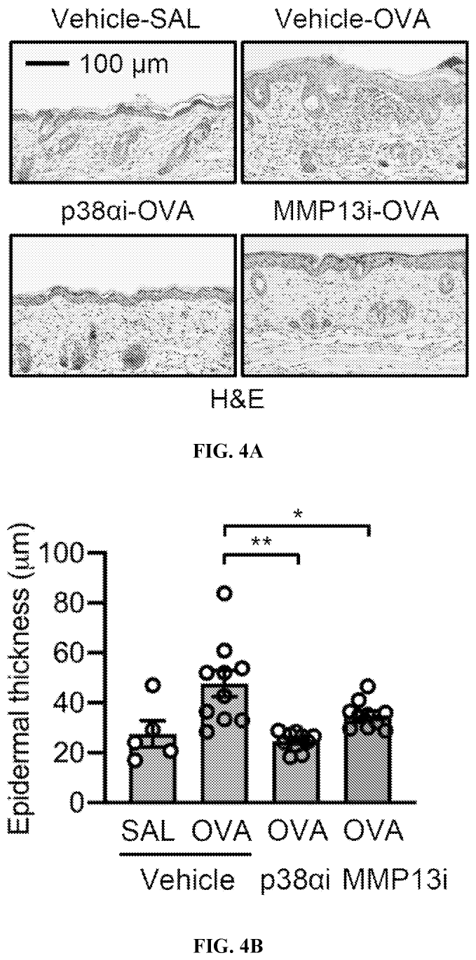

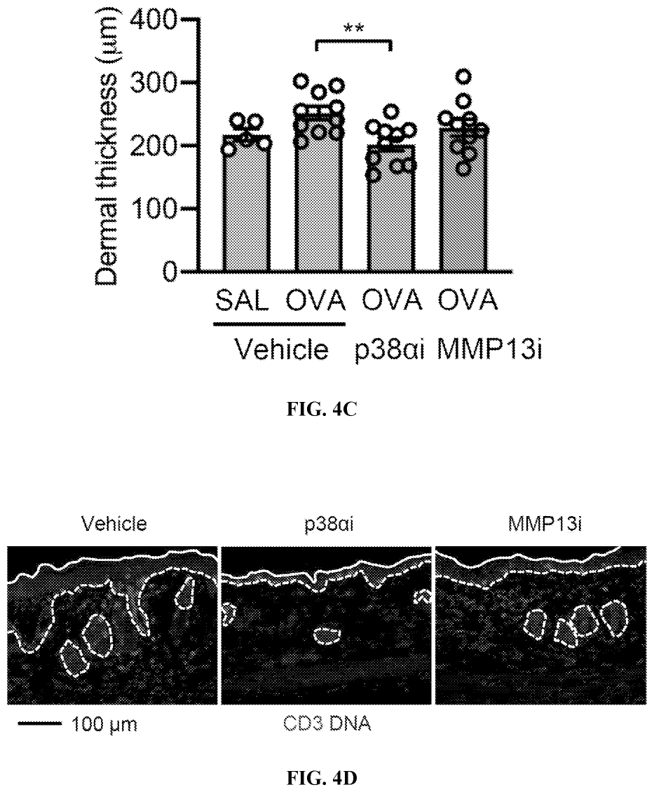

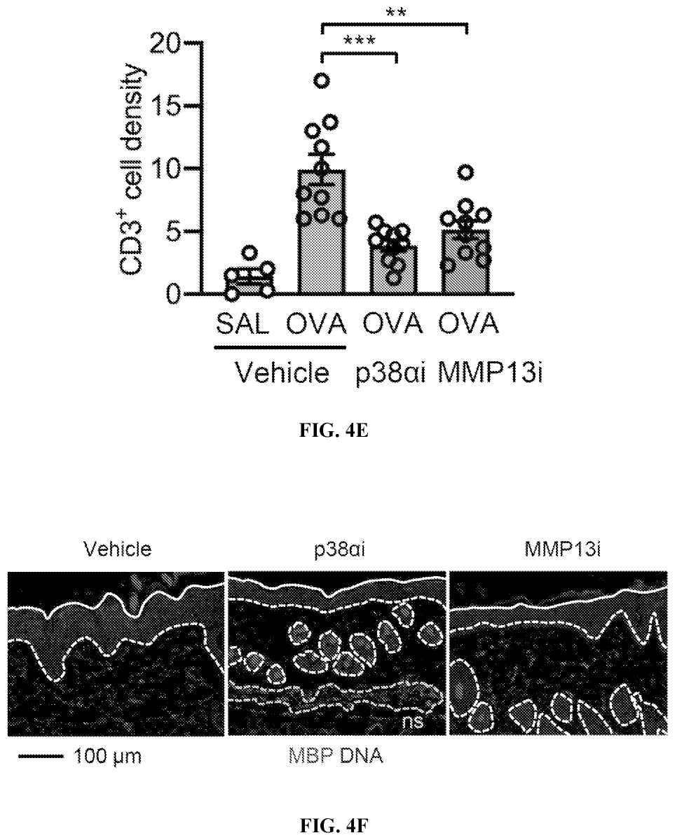

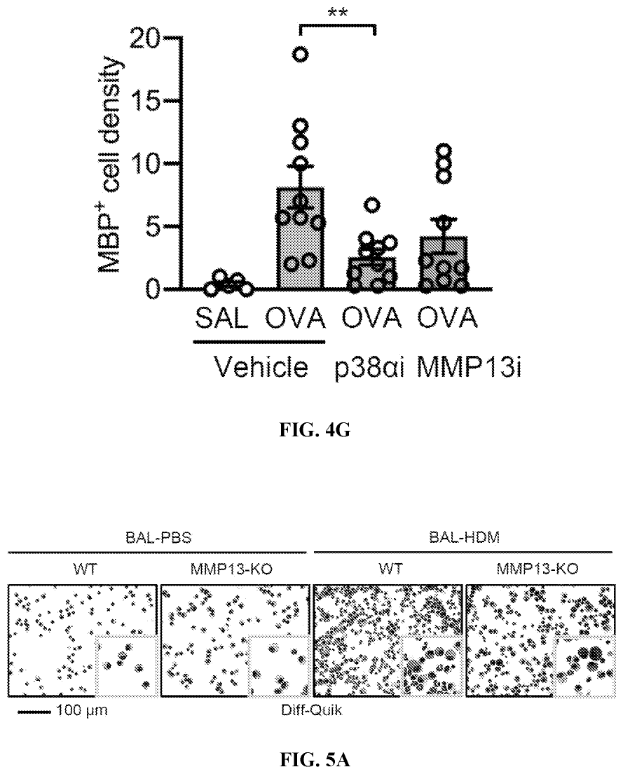

MMP13 has been implicated in inflammation-associated pathologies (Page-McCaw et al., Matrix metalloproteinases and the regulation of tissue remodelling. Nat Rev Mol Cell Biol 2007, 8, 221-233; and Khokha et al., Metalloproteinases and their natural inhibitors in inflammation and immunity. Nat Rev Immunol 2013, 13, 649-665). Its role in allergic diseases, however, has remained unexplored. Experimental data provided herein showed that MMP13 plays a key role in driving allergic skin and airway inflammation and that MMP13 is a therapeutic target for the treatment of allergic diseases. The expression and action of MMP13 in the skin epithelium make this matrix metalloproteinase a target amenable to inhibition by topically administered therapeutics. Indeed, experimental data provided herein showed that a topically applied MMP13 inhibitor was effective at suppressing allergic dermatitis in mice (see FIG. 4).

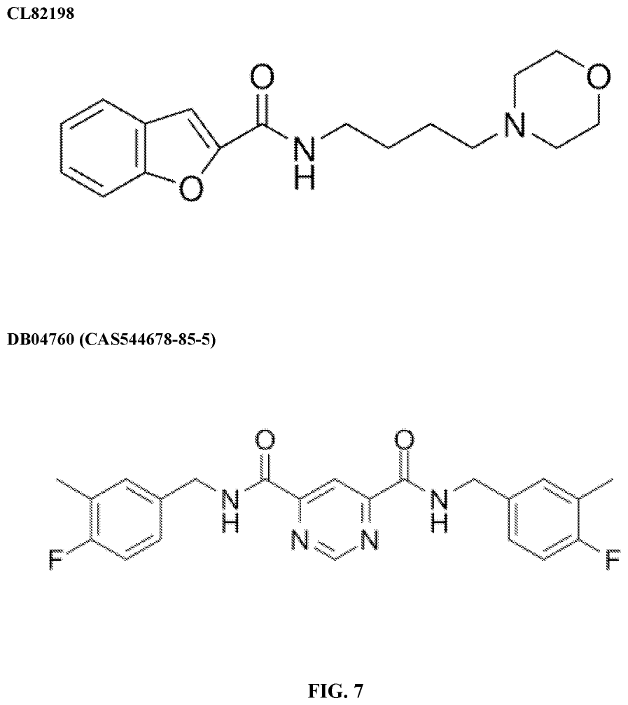

Hydroxamate-based MMP inhibitors (e.g., marimastat), which act by chelating the catalytic zinc ion and generally exhibit low target selectivity, have been shown to produce high rates of adverse effects in clinical settings (Vandenbroucke & Libert, Is there new hope for therapeutic matrix metalloproteinase inhibition? Nat Rev Drug Discov 2014, 13, 904-927). By contrast, some non-hydroxamate MMP inhibitors interact with a substrate-binding subsite, known as the “S1′ pocket,” that is separated from the zinc ion-coordinating histidine residues. X-ray crystallography and NMR spectroscopy studies revealed an unusually large S1′ pocket for MMP13 compared with those of other MMPs (Lovejoy et al., Crystal structures of MMP-1 and -13 reveal the structural basis for selectivity of collagenase inhibitors. Nat Struct Biol 1999, 6, 217-221; and Moy et al., High-resolution solution structure of the catalytic fragment of human collagenase-3 (MMP-13) complexed with a hydroxamic acid inhibitor. J Mol Biol 2000, 302, 671-689). The two MMP13 inhibitors tested in studies described herein (see FIG. 7), CL82198 (Chen et al., Structure-based design of a novel, potent, and selective inhibitor for MMP-13 utilizing NMR spectroscopy and computer-aided molecular design. J Am Chem Soc 2000, 122, 9648-9654) and DB04760 (CAS 544678-85-5; Engel et al., Structural basis for the highly selective inhibition of MMP-13. Chem Biol 2005, 12, 181-189), fill the S1′ pocket of MMP13 but cannot fit those of other MMPs. Hence, CL82198 and DB04760 (CAS 544678-85-5) are almost exclusively selective for MMP13.

Accordingly, the present disclosure provides, in some aspects, therapeutic uses of inhibitors of MMP13 for treating allergic inflammatory diseases such as atopic dermatitis and allergic asthma.

### I. Inhibitors of MMP13 and Pharmaceutical Compositions Comprising Such

Matrix metalloproteinases (MMPs) are a family of calcium-dependent zinc-containing endopeptidases that are capable of proteolytically degrading many components of the extracellular matrix. MMPs are produced in structural cells such as fibroblasts, endothelial cells, and epithelial cells as well as inflammatory cells such as macrophages, lymphocytes, neutrophils, and eosinophils. MMPs are secreted as latent forms followed by proteolytic processing to active forms.

Based on their domain organization, their sequence similarities, and the specificity of their substrates, MMPs can be classified into the following four groups: gelatinases, matrilysins, archetypal, and furin-activated. The archetypal MMPs can be classified into the following three subgroups according to their substrate specificities: collagenases, stromelysins, and other archetypal MMPs. MMP13, also known as collagenases 3, is a member of the collagenase subgroup. As used herein, MMP13 encompasses both latent and active forms of MMP13, e.g., human MMP13.

The term “inhibitor of MMP13” or “MMP13 inhibitor,” as used herein, refers to a molecule (e.g., a small molecule or a biological molecule) that blocks, inhibits, reduces (including significantly), or interferes with MMP13 (e.g., mammalian MMP13 such as human MMP13) biological activity in vitro, in situ, and/or in vivo. The term “inhibitor” implies no specific mechanism of biological action whatsoever, and expressly includes and encompasses all possible pharmacological, physiological, and biochemical interactions with MMPP13 whether direct or indirect, and whether interacting with MMP13, its substrate, or through another mechanism, and its consequences which can be achieved by a variety of different, and chemically divergent, compositions.

Non-limiting examples of an inhibitor of MMP13 for use in the methods described herein for treating an allergic inflammatory disease include a small molecule (e.g., CL82198 (CAS 307002-71-7), DB04760 (CAS 544678-85-5)), an agent that inhibits expression of MMP13 (e.g., a nucleic acid molecule that inhibit MMP13 expression such as a short interfering RNA (siRNA)), anti-MMP13 antibodies, or a peptide that inhibit MMP13 (e.g., a peptide aptamer, a MMP13 structural analog). In some embodiments, the inhibitor of MMP13 binds MMP13 (i.e., physically interacts with MMP13), binds to a substrate of MMP13, and/or inhibits expression (i.e., transcription or translation) or processing of a latent form of MMP13 into its active form.

### (a) Small Molecule Inhibitors of MMP13

Any small molecule suitable for inhibiting MMP13 can be used in methods for treating an allergic inflammatory disease as disclosed herein. The term “small molecule inhibitor of MMP13” refers to small organic compounds, inorganic compounds, or any combination thereof that inhibits or reduces MMP13 biological activity (e.g., enzymatic activity). In some embodiments, a small molecule inhibitor of MMP13 used in the methods described herein inhibits MMPP13 biological activity by at least 10% (e.g., at least 20%, at least 30%, at least 40%, at least 50%, at least 60%, at least 70%, at least 80%, at least 90%, or more).

In some examples, the small molecule inhibitor of MMP13 is a Zn²⁺-binding MMPP13 inhibitor. Without wishing to be bound by theory, zinc-binding MMP13 inhibitors bind the conserved catalytic Zn²⁺ of MMP13, thereby blocking substrate binding to MMP13 and inhibiting its activity. Non-limiting examples of Zn²⁺-binding MMP13 inhibitors include hydroxamic acid-based MMP13 inhibitors, carboxylic acid-based MMP13 inhibitors, pyrimidinetrione-based MMP13 inhibitors, triazolone-based MMP13 inhibitors, and triazole-based MMP13 inhibitors. Examples of Zn²⁺-binding MMP13 inhibitors include, but are not limited to, those disclosed in Wan et al., Selective MMP13 Inhibitors: Promising Agents for the Therapy of Osteoarthritis. Current Medicinal Chemistry, 2020, 27, 3753-3769.

In some examples, the small molecule inhibitor of MMP13 is a non-Zn²⁺-binding MMP13 inhibitor. Without wishing to be bound by theory, non-Zn²⁺-binding MMP13 inhibitors interact with a substrate-binding subsite, known as the S1′ pocket, that is separate from the zinc ion-coordinating histidine residues of MMP13, thereby blocking substrate binding to MMP13 and inhibiting its activity. Non-limiting examples of non-Zn²⁺-binding MMP13 inhibitors include furan-based MMP13 inhibitors, indole-based MMP13 inhibitors, pyrimidine-based MMP13 inhibitors, and fused pyrimidine-based MMP13 inhibitors. Examples of Zn²⁺-binding MMP13 inhibitors include, but are not limited to, those disclosed in Wan et al., Selective MMP13 Inhibitors: Promising Agents for the Therapy of Osteoarthritis. Current Medicinal Chemistry, 2020, 27, 3753-3769; and Li & Johnson, Selective MMP13 Inhibitors. Medicinal Research Reviews, 2011, 31, No. 6, 863-894.

Small molecule inhibitors of MMP13 can be used in methods described herein in the free form, as a salt thereof, or as prodrug derivatives thereof.

### (b) Other Inhibitors of MMP13

Inhibitors of MMP13 other than small molecule inhibitors described above can be used in the methods described herein. Non-limiting examples of inhibitors of MMP13 can be used in the methods described herein include agents that inhibits expression of MMP13 (e.g., nucleic acid molecules that inhibit MMP13 expression), anti-MMP13 antibodies, or peptides that inhibit MMP13.

In some examples, an inhibitor of MMP13 to be used in methods described herein can be an agent that inhibits expression of MMP13. In some embodiments, an agent that inhibits expression of MMP13 used in the methods described herein inhibits MMP13 expression by at least 10% (e.g., at least 20%, at least 30%, at least 40%, at least 50%, at least 60%, at least 70%, at least 80%, at least 90%, or more). In some examples, agents that inhibit expression of MMP13 are nucleic acid molecules such as short interfering nucleic acid (siNA), short interfering RNA (siRNA), double-stranded RNA (dsRNA), micro-RNA (miRNA), and short hairpin RNA (shRNA) molecules that bind to an MMP13 nucleic acid and inhibit expression of MMP13. Such nucleic acid molecules can include non-naturally-occurring nucleobases (e.g., modified nucleobases), sugars (e.g., substituted sugar moieties), and/or covalent internucleoside linkages (e.g., modified backbones). An exemplary sequence of a human MMP13 (also known as collagenase 3 preproprotein) nucleic acid is provided in GenBank at Acc. No. NM_002427.4. Examples of nucleic acid molecules for inhibiting expression of MMP13 include, but are not limited to, those disclosed in U.S. Pat. No. 7,667,030.

In some examples, an inhibitor of MMP13 to be used in methods described herein can be an anti-MMP13 antibody. An anti-MMP13 antibody is an antibody capable of binding to MMPP13, which can inhibit MMP13 biological activity and/or downstream pathway(s) mediated by MMP13 signaling. An exemplary sequence of a human MMP13 protein is provided in GenBank at Acc. No. NP_002418.1. In some embodiments, an anti-MMP13 antibody used in the methods described herein inhibits MMP13 biological activity and/or downstream pathway(s) mediated by MMP13 signaling by at least 10% (e.g., at least 20%, at least 30%, at least 40%, at least 50%, at least 60%, at least 70%, at least 80%, at least 90%, or more). Examples of anti-MMP13 antibodies include, but are not limited to, those disclosed in U.S. Pat. No. 8,536,313, and PCT publication WO2018220235.

An antibody is an immunoglobulin molecule capable of specific binding to a target, such as carbohydrate, polynucleotide, lipid, polynucleotide, lipid, polypeptide, through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof such as Fab, Fab′, F(ab′)₂, Fv, single chain (scFv), mutants thereof, fusion proteins comprising antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An anti-MMP13 antibody can be an antibody of any class, such as IgD, IgE, IgG, IgA, or IgM (or sub-class thereof), or the anti-MMP13 antibody need not be of any particular class.

In some examples, an inhibitor of MMP13 to be used in methods described herein can be a peptide inhibitor. For example, an inhibitor of MMP13 can be a peptide comprising a portion of a MMP13-binding protein that specifically binds to MMP13 and blocks its interaction with one or more MMP13 binding proteins. In some embodiments, a peptide inhibitor used in the methods described herein inhibits MMP13 biological activity and/or downstream pathway(s) mediated by MMP13 signaling by at least 10% (e.g., at least 20%, at least 30%, at least 40%, at least 50%, at least 60%, at least 70%, at least 80%, at least 90%, or more). Examples of peptide inhibitors of MMP13 include, but are not limited to, those disclosed in U.S. Pat. No. 9,260,707.

The human MMP13 sequences provided herein are exemplary, and other sequences with at least 80%, e.g., at least 90%, 95%, 97%, or 99% identity can be used. Calculations of identity between sequences are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

### (c) Pharmaceutical Compositions

Any of inhibitor of MMP13 (e.g., those described herein) can be mixed with a pharmaceutically acceptable excipient (carrier) to form a pharmaceutical composition for use in treating an allergic inflammatory disease (e.g., allergic asthma, atopic dermatitis). “Acceptable” means that the excipient must be compatible with the inhibitor of MMP13 (and preferably, capable of stabilizing the inhibitor of MMP13) and not deleterious to the subject to be treated. Pharmaceutically acceptable excipients (carriers), including buffers, are well known in the art. See, e.g., Remington: The Science and Practice of Pharmacy 20^{th} Ed. (2000) Lippincott Williams and Wilkins, Ed. K. E. Hoover.

Pharmaceutical compositions comprising an inhibitor of MMP13 to be used in the methods described herein can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used, and may comprise buffers (e.g., phosphate, citrate, and other organic acids); antioxidants (e.g., ascorbic acid, methionine); preservatives (e.g., octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins (e.g., serum albumin, gelatin, immunoglobulins); hydrophilic polymers (e.g., polyvinylpyrrolidone); amino acids (e.g., glycine, glutamine, asparagine, histidine, arginine, lysine); monosaccharides, disaccharides, and other carbohydrates including glucose, mannose, or dextrans; chelating agents (e.g., ETDA) sugars (e.g., sucrose, mannitol, sorbitol); salt-forming counter-ions (e.g., sodium); metal complexes (e.g., Zn-protein complexes); and/or non-ionic surfactants (e.g., TWEEN™, PLURONICS™ polyethylene glycol (PEG)).

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the inhibitor of MMPP13 with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used. Alternatively, tissue-coating solutions, such as pectin containing formulations can be used. Ophthalmic formulation, ear drops, and eye drops are also contemplated as being within the scope of this invention. Additionally, the present invention contemplates the use of transdermal patches, which have the added advantage of providing controlled delivery of an agent to the body. Such dosage forms can be made by dissolving or dispensing the inhibitor of MMP13 in the proper medium. Absorption enhancers can also be used to increase the flux of the inhibitor of MMP13 across the skin. The rate can be controlled by either providing a rate controlling membrane or by dispersing the agent in a polymer matrix or gel.

Additionally, the carrier for a topical formulation can be in the form of a hydroalcoholic system (e.g., gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids, and aqueous based mousse systems.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For preparing solid compositions such as tablets, the principal active ingredient (i.e., an inhibitor of MMP13) can be mixed with a pharmaceutical carrier, e.g., conventional tableting ingredients such as corn starch, lactose, sucrose, sorbital, talc, stearic acid, magnesium stearate, dicalcium phosphate or gums, and other pharmaceutical diluents, e.g., water, to form a solid preformulation composition containing a homogeneous mixture of an active ingredient. When referring to these preformulation compositions as homogeneous, it is meant that the active ingredient is dispersed evenly throughout the composition so that the composition can be readily subdivided into equally effective unit dosage forms such as tablets, pills, and capsules. This solid preformulation composition is then subdivided into unit dosage forms of the type described above containing from 0.1 to about 500 mg of the active ingredient of the present invention. The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coating, such materials including a number of polymeric acids and mixtures of polymeric acids with such materials as shellac, cetyl alcohol, and cellulose acetate.

Suitable surface-active agents include, but are not limited to, non-ionic agents, such as polyoxyethylenesorbitans (e.g., Tween™20, 40, 60, 80 or 85) and other sorbitans (e.g., Span™20, 40, 60, 80, or 85). Compositions with a surface-active agent can comprise between 0.05% and 5% surface-active agent (e.g., between 0.1% and 2.5%). It will be appreciated that other ingredients may be added, for example mannitol or other pharmaceutically acceptable vehicles, if necessary.

Suitable emulsions can be prepared using commercially available fat emulsions, such as Intralipid™, Liposyn™, Infonutrol™, Lipofundin™, and Lipiphysan™. The active ingredient (i.e., an inhibitor of MMP13 such as a small molecule, an anti-MMP13 antibody, a peptide inhibitor, and an agent that inhibits expression of MMP13) can be either dissolved in a pre-mixed emulsion composition or alternatively it may be dissolved in an oil (e.g., soybean oil, safflower oil, cottonseed oil, sesame oil, corn oil, or almond oil) and an emulsion formed upon mixing with a phospholipid (e.g., egg phospholipids, soybean phospholipids or soybean lecithin) and water. It will be appreciated that other ingredients can be added, for example glycerol or glucose, to adjust the tonicity of the emulsion. Suitable emulsions will typically contain up to 20% oil, for example, between 5% and 20%. The fat emulsion can comprise fat droplets between 0.1 and 1.0 μm, e.g., 0.1 and 0.5 μm, and have a pH in the range of 5.5 to 8.0.

Pharmaceutical compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as set out above. In some examples, the pharmaceutical compositions are administered by the oral or nasal respiratory route for local or systemic effect.

Compositions in preferably sterile pharmaceutically acceptable solvents can be nebulized by use of gases. Nebulized solutions can be breathed directly from the nebulizing device or the nebulizing device can be attached to a face mask, tent or intermittent positive pressure breathing machine. Solution, suspension, or powder compositions may be administered orally or nasally, from devices which deliver the formulation in an appropriate manner.

### II. Use of Inhibitors of MMP13 for Treating Allergic Inflammatory Diseases

Aspects of the present disclosure provide methods for treating allergic inflammatory disease using an inhibitor of matrix metalloproteinase 13 (MMP13). As used herein, “allergic inflammatory disease” refers to a group of immune-mediated disorders caused by an immunological reaction (e.g., an IgE-dependent immunological reaction) to an allergen (e.g., an innocuous environmental antigen). As such, allergic inflammatory diseases include allergy-mediated inflammatory conditions such as atopic dermatitis and allergic asthma. By contrast, inflammatory diseases include immune-mediated inflammatory conditions inflammatory bowel disease (IBD) and arthritis.

Non-limiting examples of allergic inflammatory disease include atopic dermatitis (also known as eczema), allergic asthma, allergic rhinitis (also known as hay fever), allergic conjunctivitis, and food allergies.

To practice the method disclosed herein, an effective amount of a composition comprising an inhibitor of MMP13 (e.g., a pharmaceutical composition comprising an inhibitor of MMP13) can be administered to a subject (e.g., a human patient) having or at risk for having an allergic inflammatory disease via a suitable route (e.g., topical administration, inhalation).

The term “subject” refers to a subject who needs treatment as described herein. In some embodiments, the subject is a human (e.g., a human patient) or a non-human mammal (e.g., cat, dog, horse, cow, goat, or sheep). A human subject who needs treatment can be a human patient having, suspected of having, or at risk for having an allergic inflammatory disease, e.g., atopic dermatitis, asthma, allergic rhinitis, or allergic conjunctivitis. A subject having an allergic inflammatory disease can be identified by routine medical examination, e.g., medical examination (e.g., history and physical), laboratory tests (e.g., blood tests), imaging tests (e.g., CT scans), or skin prick testing. Such a subject can exhibit one or more symptoms associated with an allergic inflammatory disease, e.g., rashes, dry skin, itching, difficulty breathing, chest pain, cough, wheezing, sneezing, congestion, loss of smell, runny nose, watery eyes, redness, watery eyes, puffy eyes, or a combination thereof. Alternatively or in addition, such a subject can have one or more risk factors for an allergic inflammatory disease, e.g., family history, viral infections (e.g., viral respiratory infections), genetic factors, occupational exposures, environmental exposures, smoking, air pollution, and obesity.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or virtually any other reason.

Empirical considerations such as the half-life of an agent will generally contribute to the determination of the dosage. Frequency of administration can be determined and adjusted over the course of therapy, and is generally, but not necessarily, based on treatment and/or suppression and/or amelioration and/or delay of an allergic inflammatory disease (e.g., atopic dermatitis, asthma, allergic rhinitis, and allergic conjunctivitis). Alternatively, sustained continuous release formulations of therapeutic agent may be appropriate. Various formulations and devices for achieving sustained release are known in the art.

In some embodiments, dosages of an inhibitor of MMP13 as described herein can be determined empirically in individuals who have been given one or more administration(s) of the inhibitor of MMP13. For example, individuals are given incremental dosages of the inhibitor of MMP13, and an indicator and/or a symptom of an allergic inflammatory disease can be followed to assess efficacy of the inhibitor of MMP13.

Generally, for administration of any of the inhibitors of MMP13 such as those described herein, an initial candidate dosage can be about 2 mg/kg. For example, a typical daily dosage can range from about any of 0.1 μg/kg to 3 μg/kg to 30 μg/kg to 300 μg/kg to 3 mg/kg to 30 mg/kg to 100 mg/kg or more, depending on factors described herein. For repeated administrations over several days or longer, depending on the condition, the treatment can be sustained until a desired suppression of symptoms occurs or until sufficient therapeutic levels are achieved to alleviate an allergic inflammatory disease, or a symptom thereof. In such instances, a dosing regimen can comprise administration of an initial dose, followed by a weekly maintenance dose, or followed by a maintenance dose every other week.

Any suitable dosing regimen can be used in methods described herein. In some embodiments, the dosage regimen depends on the pattern of pharmacokinetic decay that the practitioner wishes to achieve. In some embodiments, dosing from one to four times per week can be used. In some embodiments, dosing from about 3 μg/kg to about 2 mg/kg (e.g., about 3 μg/kg, about 10 μg/kg, about 30 μg/kg, about 100 μg/kg, about 300 μg/kg, about 1 mg/kg, and about 2 mg/kg) can be used. In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks, or once every month, every 2 months, or every 3 months, or longer. In some embodiments, dosing regimens (including inhibitor used) can vary over time.

In some embodiments, the inhibitor of MMP13 is administered at a dose of about 0.001 mg to about 200 mg a day. For example, the inhibitor of MMP13 is administered at a dose of about 0.01 mg to about 100 mg a day, about 0.01 mg to about 50 mg a day about 0.01 mg to about 10 mg a day, or about 0.1 mg to about 10 mg a day.

In some embodiments, when the inhibitor of MMP13 is administered topically, the inhibitor of MMP13 can be administered at a dose of about 0.001 mg of the inhibitor of MMP13 per m² of skin surface to about 200 mg of the inhibitor of MMP13 per m² of skin surface a day. For example, the inhibitor of MMP13 is administered topically at a dose of about 0.01 mg/m² to about 100 mg/m² a day, about 0.01 mg/m² to about 50 mg/m² a day about 0.01 mg/m² to about 10 mg/m² a day, or about 0.1 mg/m² to about 10 mg/m² a day. In some examples, the inhibitor of MMP13 is applied topically more than once per day, e.g., 2, 3, 4, 5, or more administrations per day.

In some embodiments, the appropriate dosage of an inhibitor of MMP13 will depend on the specific inhibitor(s) (or pharmaceutical compositions thereof) used, the type and severity of allergic inflammatory disease(s), previous therapy, the patient's clinical history and response to the inhibitor(s), and the discretion of the healthcare practitioner.

As used herein, the term “treating” refers to the application or administration of a composition including one or more active agents to a subject who has an allergic inflammatory disease (e.g., atopic dermatitis, asthma, allergic rhinitis, and allergic conjunctivitis), a symptom of an allergic inflammatory disease, and/or a predisposition toward an allergic inflammatory disease, with the purpose to cure, heal, alleviate, relieve, alter, remedy, ameliorate, improve, or affect the allergic inflammatory disease, the symptom of the allergic inflammatory disease, and/or the predisposition toward the allergic inflammatory disease.

Alleviating an allergic inflammatory disease includes delaying the development or progression of the disease, and/or reducing disease severity. Alleviating the disease does not necessarily require curative results.

As used herein, “delaying” the development of an allergic inflammatory disease (e.g., atopic dermatitis, asthma, allergic rhinitis, and allergic conjunctivitis) means to defer, hinder, slow, retard, stabilize, and/or postpone progression of the allergic inflammatory disease. This delay can be of varying lengths of time, depending on the history of the allergic inflammatory disease and/or individuals being treated. A method that “delays” or alleviates the development of an allergic inflammatory disease and/or delays the onset of the allergic inflammatory disease is a method that reduces probability of developing one or more symptoms of the allergic inflammatory disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the allergic inflammatory disease. Development of the allergic inflammatory disease can be detectable and assessed using standard clinical techniques known in the art. However, development also refers to progression that may be undetectable. For purposes of this disclosure, development or progression refers to the biological course of the symptoms. “Development” includes occurrence, recurrence, and onset. As used herein, “onset” or “occurrence” of an allergic inflammatory disease includes initial onset and/or recurrence.

In some embodiments, the inhibitor of MMP13 is administered to a subject in an amount sufficient to reduce levels of MMP13-mediated signaling by at least 10% (e.g., at least 20%, at least 30%, at least 40%, at least 50%, at least 60%, at least 70%, at least 80%, at least 90%, or more).

In some embodiments, the inhibitor of MMP13 is administered to a subject in an amount sufficient to reduce levels of MMP13 biological activity by at least 10% (e.g., at least 20%, at least 30%, at least 40%, at least 50%, at least 60%, at least 70%, at least 80%, at least 90%, or more).

In some embodiments, the inhibitor of MMP13 is administered to a subject in an amount sufficient to reduce levels of MMP13 (e.g., MMP13 protein and/or nucleic acids) by at least 10% (e.g., at least 20%, at least 30%, at least 40%, at least 50%, at least 60%, at least 70%, at least 80%, at least 90%, or more).

In some embodiments, the inhibitor of MMP13 is administered to a subject in an amount sufficient to reduce levels of inflammation by at least 10% (e.g., at least 20%, at least 30%, at least 40%, at least 50%, at least 60%, at least 70%, at least 80%, at least 90%, or more).

In some embodiments, the inhibitor of MMP13 is administered to a subject in an amount sufficient to reduce epidermal thickening by at least 10% (e.g., at least 20%, at least 30%, at least 40%, at least 50%, at least 60%, at least 70%, at least 80%, at least 90%, or more).

In some embodiments, the inhibitor of MMP13 is administered to a subject in an amount sufficient to reduce infiltration of immune cells (e.g., T cells and/or eosinophils) by at least 10% (e.g., at least 20%, at least 30%, at least 40%, at least 50%, at least 60%, at least 70%, at least 80%, at least 90%, or more).

In some embodiments, the inhibitor of MMP13 is administered to a subject in an amount sufficient to reduce edema formation by at least 10% (e.g., at least 20%, at least 30%, at least 40%, at least 50%, at least 60%, at least 70%, at least 80%, at least 90%, or more).

The inhibitors of MMP13 can be administered using any suitable method for achieving delivery of the inhibitor to the subject in need thereof. The route of administration can depend on various factors such as the type of allergic inflammatory disease to be treated and the site of the disease. In some embodiments, the inhibitor of MMP13 can be administered nasally, topically, parenterally, buccally, or by inhalation. Parenteral administration includes, but is not limited to, subcutaneous, intracutaneous, intravenous, intramuscular, or intrasynovial injection or infusion techniques.

The particular dosage regimen, e.g., dose, timing, and repetition, used in methods described herein will depend on the particular subject and that subject's medical history.

In some embodiments, more than one inhibitor of MMP13 can be administered to a subject in need thereof (e.g., a small molecule inhibitor and a peptide inhibitor are administered to the subject). The inhibitor of MMP13 can be the same type or different from each other. At least one, at least two, at least three, at least four, or at least five different inhibitors of MMP13 can be co-administered. In such instances, inhibitors of MMP13 can have complementary activities that do not adversely affect each other. Inhibitors of MMP13 can also be used in conjunction with other agents that serve to enhance and/or complement the effectiveness of the inhibitor.

In some embodiments, the inhibitor of MMP13 is administered one or more times to the subject. Alternatively, or in addition to, the inhibitor of MMP13 can be administered as part of a combination therapy comprising an inhibitor of MMP13 (e.g., CL82198 (CAS 307002-71-7) or DB04760 (CAS 544678-85-5)) and an additional therapeutic agent.

Any therapeutic agent suitable for treating allergic inflammatory disease can be used as an additional therapeutic agent in methods and/or compositions described herein. Non-limiting examples of additional therapeutic agents include anti-inflammatory agents (e.g., nonsteroidal anti-inflammatory drugs (NSAIDs) such as calcineurin inhibitors (TCIs), steroids such as corticosteroids), antihistamines (e.g., levocabastine, emedastine difumarate), and immunosuppressants (e.g., ciclosporin, azathioprine, methotrexate, mycophenolate mofetil), IL-4Rα neutralizing agents (e.g., IL-4Rα neutralizing antibodies such as dupilumab), and JAK inhibitors (e.g., upadacitinib, baricitinib, tofacitinib). Alternatively, in some embodiments no other agents are used.

The term combination therapy, as used herein, embraces administration of these agents in a sequential manner, that is wherein each therapeutic agent is administered at a different time, as well as administration of these therapeutic agents, or at least two of the agents, in a substantially simultaneous manner.

Sequential or substantially simultaneous administration of each agent can be affected by any appropriate route including, but not limited to, oral routes, intravenous routes, intramuscular routes, subcutaneous routes, and direct absorption through mucous membrane tissues. The agents can be administered by the same route or by different routes. For example, a first agent can be administered orally, and a second agent can be administered intravenously.

As used herein, the term “sequential” means, unless otherwise specified, characterized by a regular sequence or order, e.g., if a dosage regimen includes the administration of a first therapeutic agent and a second therapeutic agent, a sequential dosage regimen could include administration of the first therapeutic agent, before, simultaneously, substantially simultaneously, or after administration of the second therapeutic agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents described herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any way as limiting their scope.

### Materials and Methods

The following materials and methods were used in the Examples set forth herein.

### Mice

p38α-EKO mice were generated by crossing mice harboring floxed p38α alleles (Mapk14^{tm1.2Otsu}; Nishida K. et al., p38α mitogen-activated protein kinase plays a critical role in cardiomyocyte survival but not in cardiac hypertrophic growth in response to pressure overload. Mol Cell Biol 24, 10611-10620 (2004)) with mice carrying a Cre recombinase transgene expressed under the control of the human KRT14 promoter (Tg[KRT14-cre]8Brn; Jonkers J. et al., Synergistic tumor suppressor activity of BRCA2 and p53 in a conditional mouse model for breast cancer. Nat Genet 29, 418-425 (2001)). MMP13-KO (Mmp13^{-/-}; Mmp13^{tm1Smk}) mice had a systemic deficiency of MMP13 (Inada M. et al., Critical roles for collagenase-3 (Mmp13) in development of growth plate cartilage and in endochondral ossification. Proc Natl Acad Sci USA 101, 17192-17197 (2004)). MMP13-EKO mice were generated by crossing mice with floxed MMP13 alleles (Mmp13^{tm1Werb}; Stickens D. et al., Altered endochondral bone development in matrix metalloproteinase 13-deficient mice. Development 131, 5883-5895 (2004)) with mice carrying Tg[KRT14-cre]1Amc/J alleles (Dassule H. R. et al., Sonic hedgehog regulates growth and morphogenesis of the tooth. Development 127, 4775-4785 (2000)). These mice were in a C57BL/6J background and maintained in specific pathogen-free conditions. All animal experiments were conducted under an Institutional Animal Care and Use Committee-approved protocol.

### Primary Cells and Cell Lines

Primary human keratinocytes (American Type Culture Collection) in a proliferative state were cultured in keratinocyte-SFM medium (Thermo Fisher Scientific). To prepare differentiated keratinocytes, proliferative keratinocyte populations were cultured in the presence of calcium chloride (1.2 mM) for five days. HaCaT cells (American Type Culture Collection) were cultured in Dulbecco's modified Eagle's medium containing supplemented with 10% fetal bovine serum and penicillin-streptomycin (50 unit/ml and 50 μg/ml, respectively; all from Thermo Fisher Scientific).

### Small-Molecule Compounds

SCIO469 (Sigma) was dissolved in water. CL82198 (Sigma) and DB04760 (EMD Millipore) were dissolved in 50% dimethyl sulfoxide (DMSO).

### OVA-Induced Allergic Dermatitis

For allergen sensitization, OVA (Sigma) and alum adjuvant (Imject; Thermo Fisher Scientific) were combined in saline (0.9% sodium chloride; SAL) to constitute an inoculum (1 mg/ml and 100 mg/ml, respectively) and injected intraperitoneally into mice (200 μl per animal) on day 0 and 7. To elicit dermatitis in OVA-sensitized mice, shaved dorsal skin was subjected to 15 strokes of tape stripping (Tegaderm; 3M) and then challenged with OVA on days 14, 16 and 19. For this epicutaneous challenge, 100 μl of OVA (1 mg/ml in SAL) was applied to cotton gauze (1 cm×1 cm) placed on tape-stripped skin; 100 μl of SAL was applied as a control. For topical administration of p38α and MMP13 inhibitors, 125 μl of inhibitor solution (50 mM) was applied to skin sites concurrently with SAL or OVA. Challenged skin was sampled on day 21 for analysis.

### TPA-Induced Acute Dermatitis

To induce acute skin inflammation, shaved dorsal skin was treated with 200 μl of TPA (50 μg/ml in acetone) daily for 2 consecutive days; inflamed skin was sampled 2 days later for analysis.

### HDM-Induced Allergic Airway Inflammation

Mice were subjected to intranasal instillation of HDM (D. farinae) extract (XPB81D3A25; Stallergenes Greer) in phosphate-buffered saline (PBS) on days 0, 2, 5, 7, 9, 12, 14, 16 and 19. The amounts of HDM extract administered range between 1 μg and 10 μg per animal; the administered amount for each batch was determined by seeking a condition for BAL eosinophils of 2-5×10⁵ cells. PBS was instilled as a control. Dexamethasone and MMP13 inhibitors were injected intraperitoneally (1 mg/kg and 50 mg/kg, respectively) on days 19 and 20. BAL and lungs of PBS- and HDM-challenged mice were prepared on day 21 for analysis.

### Histology and Immunofluorescence

Mouse skin and lung tissues were fixed with formalin and embedded in paraffin. Tissue sections were analyzed by hematoxylin and eosin (H&E) and Periodic acid-Schiff (PAS) staining or by immunofluorescence analysis using primary antibodies in conjunction with Alexa Fluor 594- or fluorescein isothiocyanate-conjugated secondary antibodies (Thermo Fisher Scientific) and Hoechst 33342 (Sigma). Primary antibodies specific to the following antigens were used in immunofluorescence analysis after 1:100 to 1:200 dilution: CD3 (SP7; Abcam), MBP (2000-124; Mayo Clinic), and MMP13 (ab39012; Abcam). Cytospins of BAL cells were analyzed by Diff-Quik staining.

### CRISPR Genome Editing

HaCaT cell clones with deletions in the p38α gene (MAPK14) were generated by CRISPR genome editing using a method described previously (Luo J. et al. Speed genome editing by transient CRISPR/Cas9 targeting and large DNA fragment deletion. J Biotechnol 281, 11-20 (2018)).

### Gene Expression Analysis

For transcriptome analysis, total RNA was isolated using the Trizol Reagent (Thermo Fisher Scientific), further purified using the RNeasy Mini Kit (Qiagen), and subjected to sequencing library construction (TruSeq Stranded mRNA Prep, Illumina) and 50-cycle single-end sequencing (NextSeq 550, Illumina) at the Cutaneous Biology Research Center of MGH. Sequencing reads were mapped to a Homo sapiens reference genome (assembly hg19) using the STAR genome alignment algorithm. Read counts were calculated using the HOMER scripts analyzeRepeats.pl and getDiffExpression.pl with implementation of DESeq2 through R. For qPCR analysis, total RNA isolated using the Trizol Reagent were subjected to cDNA synthesis using the SuperScript IV VILO Master Mix (Thermo Fisher Scientific) and PCR using the SYBR Green PCR Master Mix (Applied Biosystems) and gene-specific primers. Heat maps of gene transcript abundances were generated using the Morpheus software (Broad Institute).

### Statistical Analysis

Data values are expressed as means±SEM. P values were obtained with the unpaired two-tailed Student t test with Welch's correction.

### Example 1: Role of Epithelial p38α Signaling in Allergic Dermatitis

Targeted disruption of the p38α gene in mice results in embryonic lethality (Tamura K. et al., Requirement for p38α in erythropoietin expression: a role for stress kinases in erythropoiesis. Cell 102, 221-231 (2000); Adams R. H. et al., Essential role of p38α MAP kinase in placental but not embryonic cardiovascular development. Mol Cell 6, 109-116 (2000); Allen M. et al., Deficiency of the stress kinase p38α results in embryonic lethality: characterization of the kinase dependence of stress responses of enzyme-deficient embryonic stem cells. J Exp Med 191, 859-870 (2000); and Mudgett J. S. et al., Essential role for p38α mitogen-activated protein kinase in placental angiogenesis. Proc Natl Acad Sci USA 97, 10454-10459 (2000)), precluding the study of its role in adult tissues.

To circumvent this problem, we generated and characterized a panel of cell type-specific p38α-knockout (KO) mice (Kim C. et al., The kinase p38α serves cell type-specific inflammatory functions in skin injury and coordinates pro- and anti-inflammatory gene expression. Nat Immunol 9, 1019-1027 (2008); Ritprajak P. et al., Cell type-specific targeting dissociates the therapeutic from the adverse effects of protein kinase inhibition in allergic skin disease. Proc Natl Acad Sci USA 109, 9089-9094 (2012); Caballero-Franco C. et al., Tuning of protein kinase circuitry by p38α is vital for epithelial tissue homeostasis. J Biol Chem 288, 23788-23797 (2013); and Hayakawa M. et al., Loss of Functionally Redundant p38 Isoforms in T Cells Enhances Regulatory T Cell Induction. J Biol Chem 292, 1762-1772 (2017)).

Previous studies found that mice with keratinocyte-restricted p38α gene deficiency (Mapk14^{fl/fl}-KRT14Cre) exhibited attenuated skin inflammation upon exposure to ultraviolet-B radiation (Sano Y. & Park J. M. Loss of Epidermal p38α signaling prevents ultraviolet radiation-induced inflammation via acute and chronic mechanisms. J Invest Dermatol 134, 2231-2240 (2014)) and were resistant to skin blistering disease triggered by pathogenic autoantibody injection (Mao X. et al., p38 MAPK activation is downstream of the loss of intercellular adhesion in pemphigus vulgaris. J Biol Chem 286, 1283-1291 (2011)).

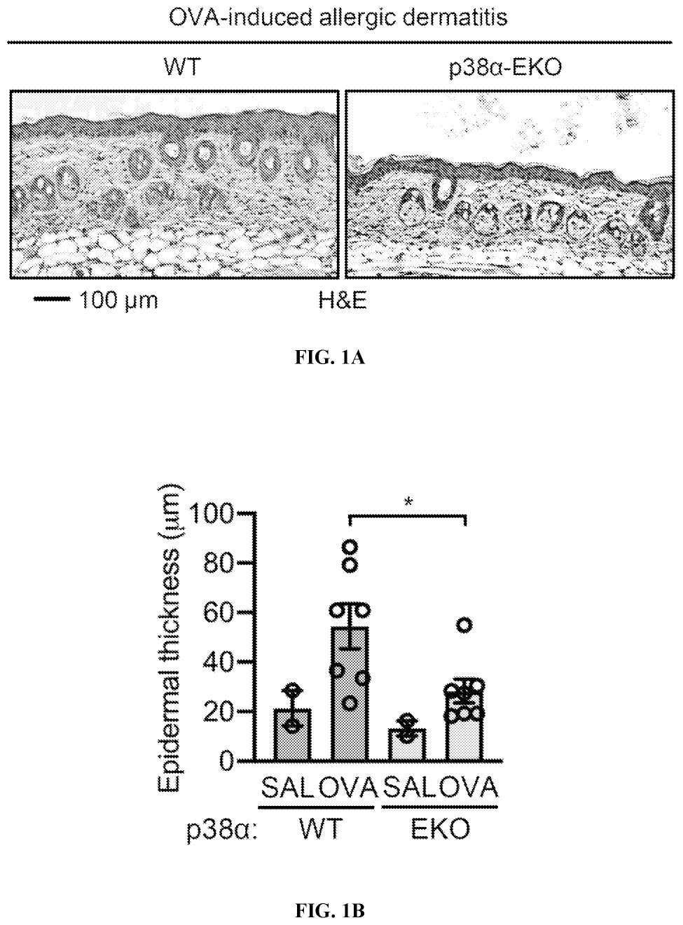

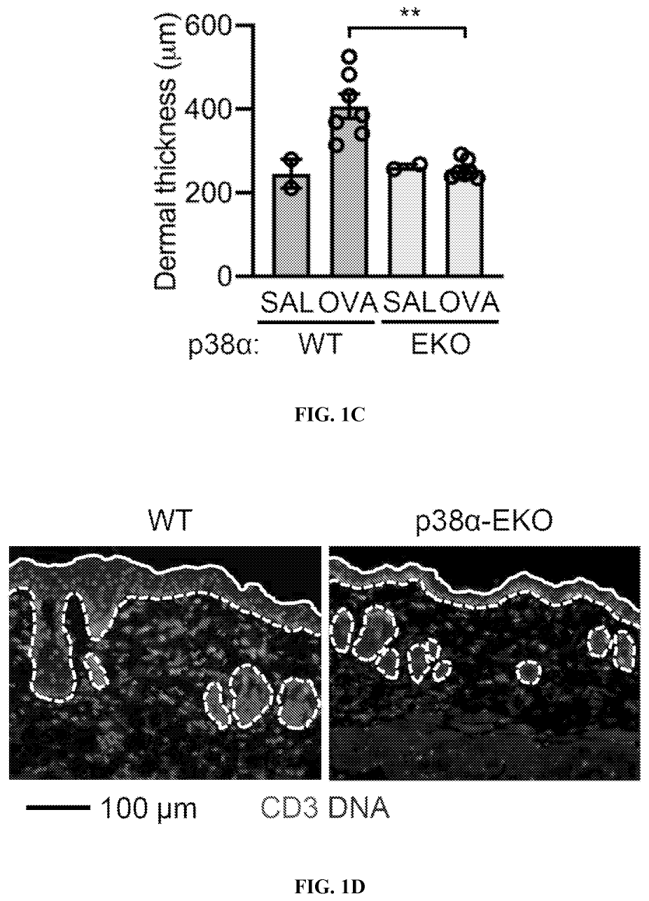

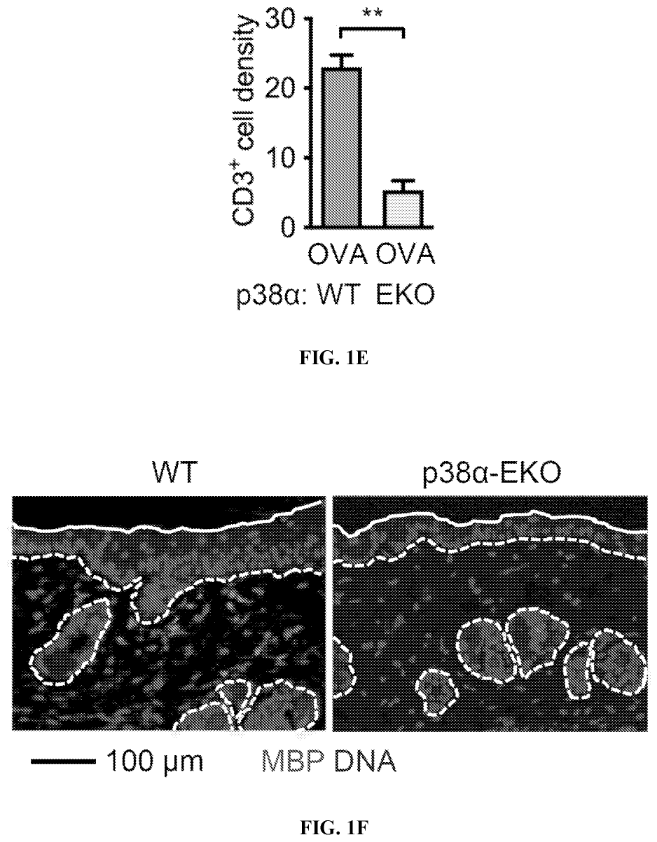

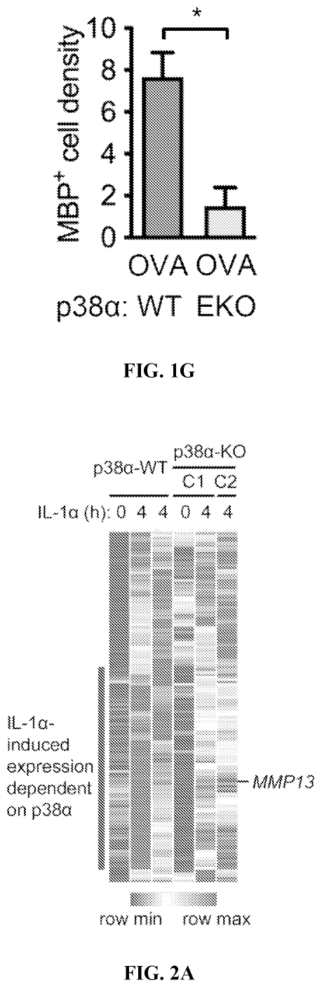

Studies described herein examined how these epithelial-specific p38α-knockout (p38α-EKO) mice respond to the experimental allergen ovalbumin (OVA) in a protocol that induces atopic dermatitis-like disease. This protocol involves intraperitoneal immunization for sensitization to allergen and epicutaneous allergen challenge on tape-stripped skin for the elicitation of dermatitis. p38α-EKO mice exhibited a marked decrease in OVA-induced epidermal and dermal thickening, indicative of acanthosis and edema formation, respectively, relative to wild-type (WT) mice (FIGS. 1A-1C). T cell and eosinophil infiltration in the OVA-challenged skin site, detected by CD3 and major basic protein (MBP) immunostaining, respectively, was also diminished in p38α-EKO mice (FIGS. 1D-1G).

These findings indicated a crucial role for epithelial p38α signaling in inflammation and tissue alteration in allergen-exposed skin.

### Example 2: MMP13 as a Mediator of p38α-Driven Allergic Dermatitis

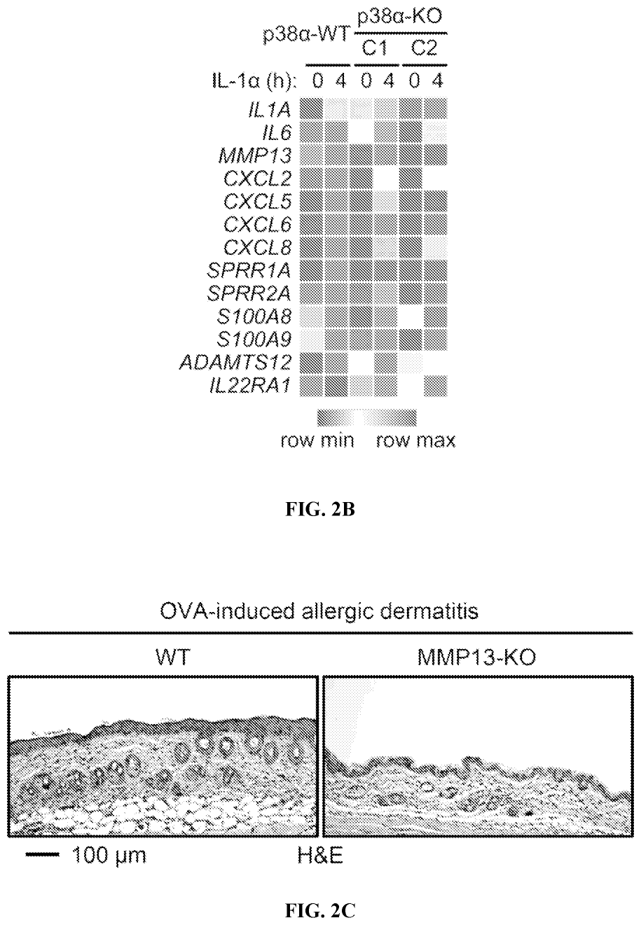

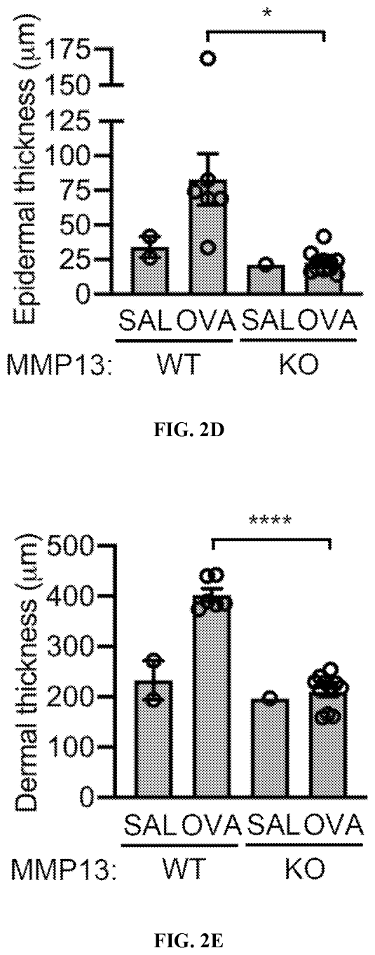

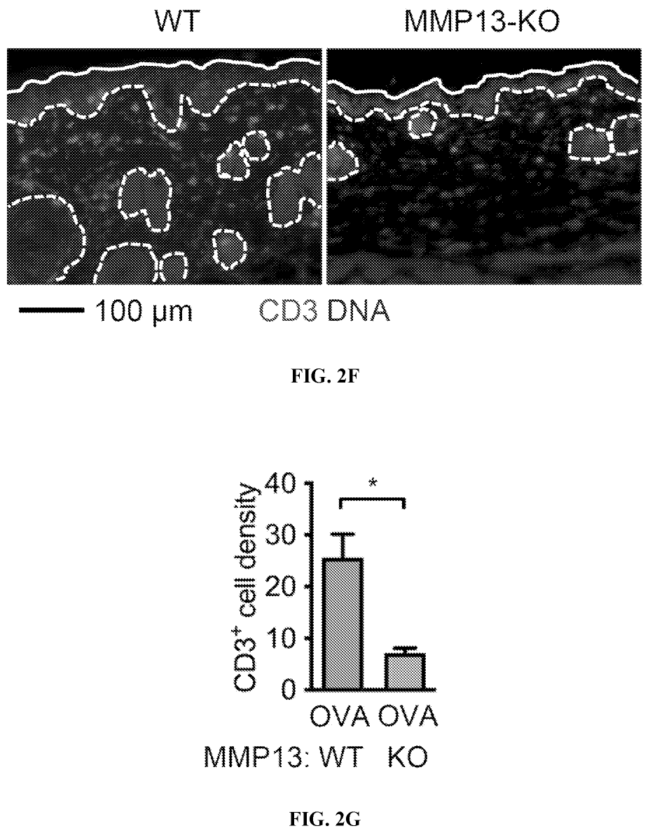

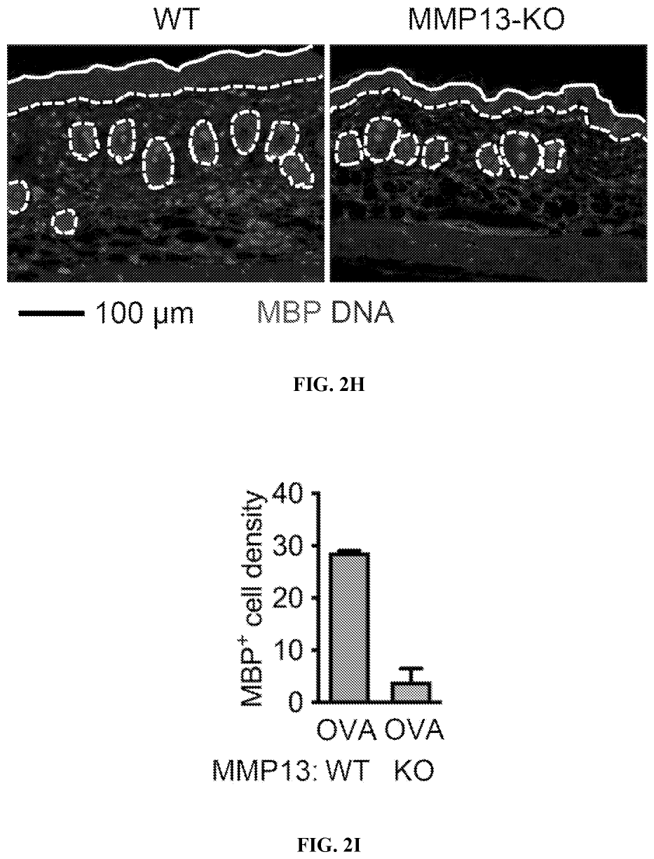

p38α signaling contributes to stimulus-induced changes in cell function by altering gene expression (Arthur J. S. & Ley S. C. Mitogen-activated protein kinases in innate immunity. Nat Rev Immunol 13, 679-692 (2013)). To identify the molecular events that are downstream to and mediate the inflammation-driving function of p38α, gene expression in control and CRISPR-engineered p38α-KO HaCaT keratinocytes was compared by RNA sequencing (RNA-seq) analysis. These cells were left unstimulated or stimulated with IL-1α, a pro-inflammatory cytokine and potent inducer of p38α, before RNA isolation. This transcriptome analysis revealed a set of genes whose induction by IL-1α was dependent on p38α (FIG. 2A). These p38α-dependent genes included many that were known to serve inflammation-related functions. Among the inflammatory mediators identified as targets of p38α signaling in RNA-seq analysis and validated by quantitative polymerase chain reaction (qPCR) analysis (FIG. 2B) was the matrix metalloproteinase MMP13.

MMP13 has been implicated in the pathogenesis of osteoarthritis and rheumatoid arthritis (Takaishi H. et al., Joint diseases and matrix metalloproteinases: a role for MMP-13. Curr Pharm Biotechnol 9, 47-54 (2008); Li N. G. et al., New hope for the treatment of osteoarthritis through selective inhibition of MMP-13. Curr Med Chem 18, 977-1001 (2011); and Xie X. W. et al., Recent Research Advances in Selective Matrix Metalloproteinase-13 Inhibitors as Anti-Osteoarthritis Agents. Chem Med Chem 12, 1157-1168 (2017)).

Many MMP13 inhibitors have been developed for the treatment of these diseases (Xie X. W. et al., Recent Research Advances in Selective Matrix Metalloproteinase-13 Inhibitors as Anti-Osteoarthritis Agents. Chem Med Chem 12, 1157-1168 (2017); Li, J. J. & Johnson, A. R. Selective MMP13 inhibitors. Med Res Rev 31, 863-894 (2011); and Vandenbroucke, R. E. & Libert, C. Is there new hope for therapeutic matrix metalloproteinase inhibition? Nat Rev Drug Discov 13, 904-927 (2014)). Its role and potential for therapeutic targeting in allergic diseases, however, have remained unexplored.

To determine the role of MMP13 in allergic inflammation, OVA-induced allergic dermatitis in WT versus MMP13-KO mice was examined. MMP13 deficiency significantly reduced acanthosis and edema formation (FIGS. 2C-2E) as well as T cell infiltration (FIGS. 2F-2G) in the OVA-challenged skin. MMP13-KO mice also exhibited a trend of reduction (P=0.0618) in OVA-induced eosinophil infiltration (FIGS. 2H-2I).

These results demonstrated that MMP13 functioned downstream of p38α and served as a mediator of p38α-driven allergic dermatitis.

### Example 3: Allergic Dermatitis Driven by Epithelial-Derived MMP13

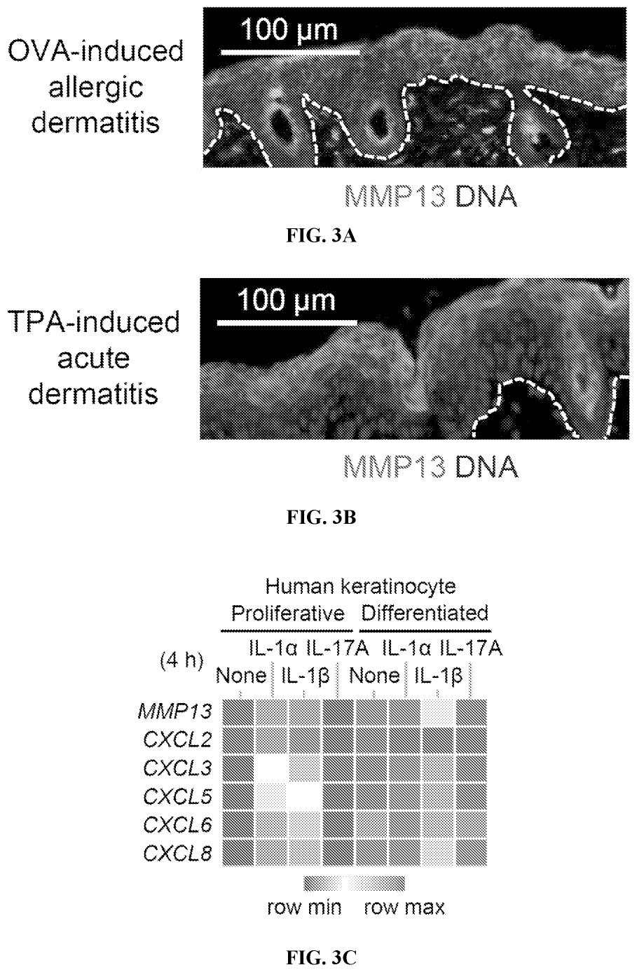

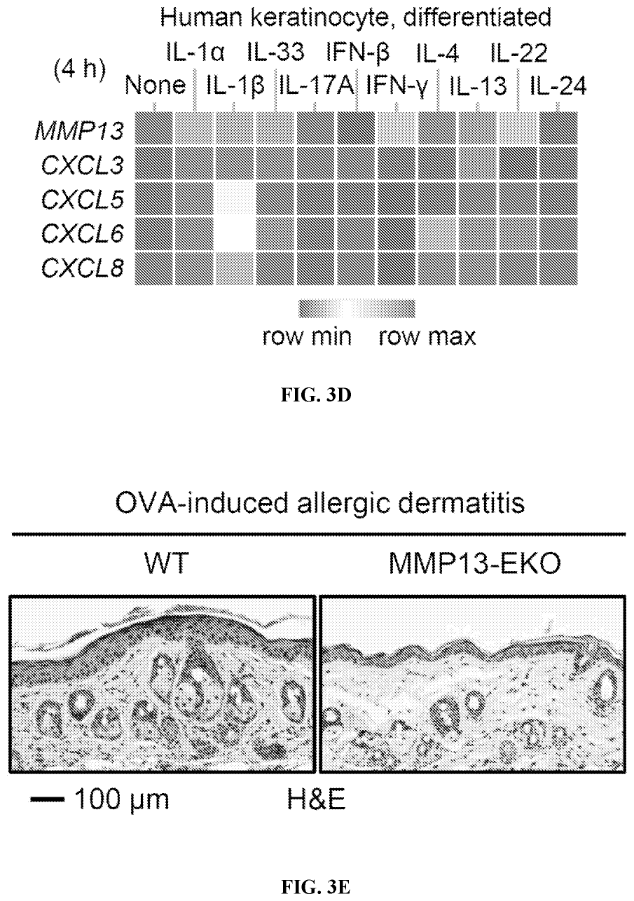

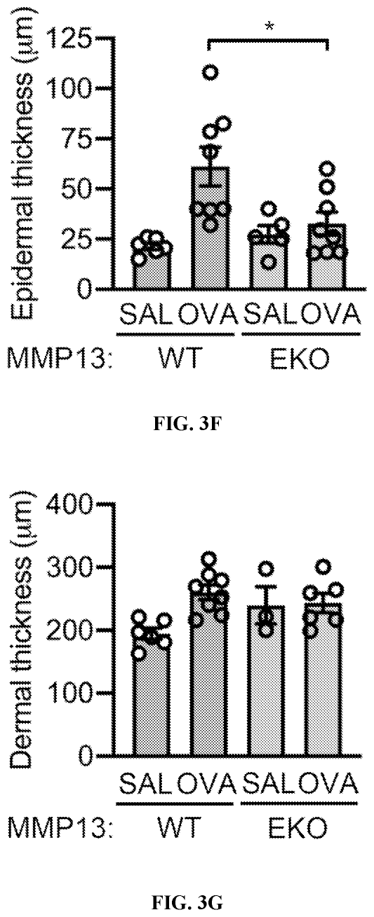

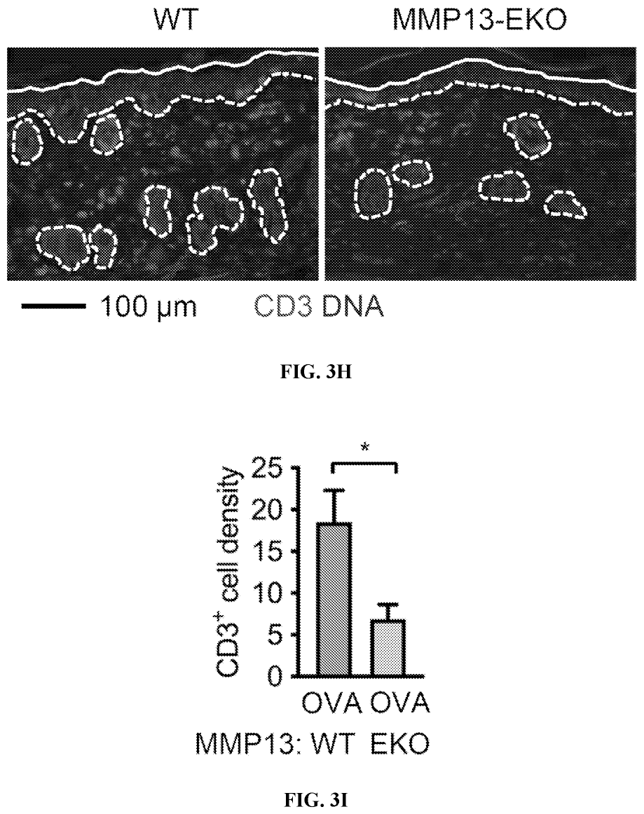

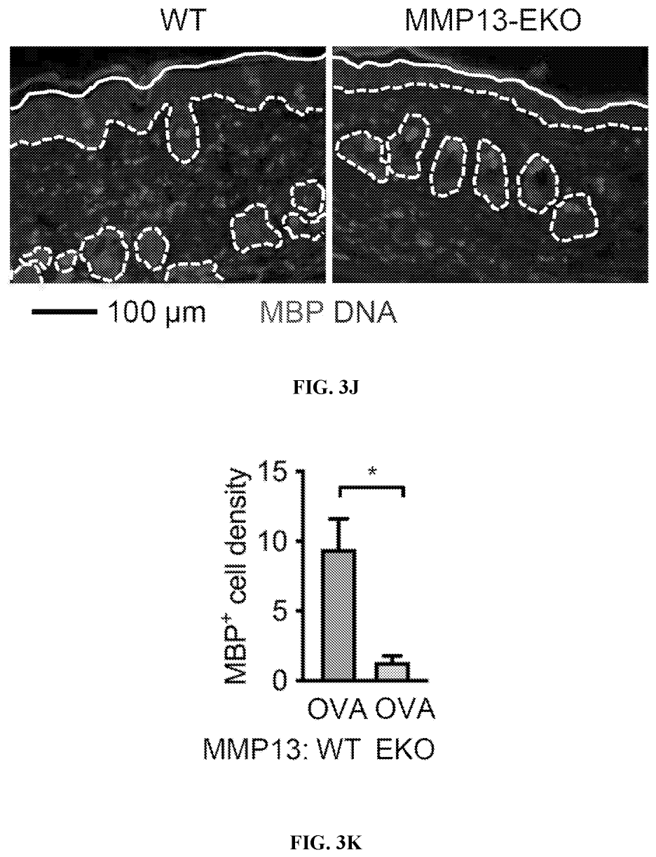

Immunofluorescence analysis revealed that the expression of MMP13 in OVA-challenged inflamed skin was confined to the suprabasal epidermal layer (FIG. 3A). Suprabasal epidermal MMP13 expression was also observed in mouse skin painted with 12-O-tetradecanoylphorbol-13 acetate (TPA), a chemical irritant inducing acute dermatitis (FIG. 3B). The basal and suprabasal epidermal layers comprise undifferentiated and proliferative keratinocytes and differentiated keratinocytes, respectively. MMP13 expression was more strongly induced by IL-1α and IL-1β in differentiated human keratinocytes than in proliferative counterparts (FIG. 3C). In addition to the two IL-1 isoforms, IL-4, a cytokine functionally linked to Th2 immune responses, enhanced MMP13 expression in differentiated human keratinocytes (FIG. 3D).

To determine the effects of ablating MMP13 expression specifically in the skin epithelium, mice were generated in which loxP-flanked (floxed) MMP13 alleles were deleted by Cre recombinase only in keratinocytes (Mmp13^{fl/fl}-KRT14Cre; MMP13-EKO). Similar to mice with systemic MMP13 gene deficiency (MMP13-KO), MMP13-EKO mice displayed attenuated allergic dermatitis when subjected to the OVA sensitization and challenge protocol (FIGS. 3E-3K).

Taken together, these results demonstrate that loss of epithelial MMP13 function is sufficient to suppress allergic dermatitis.

### Example 4: Effects of Pharmacological Inhibition of p38α and MMP13 in Allergic Dermatitis

The findings from the genetic analyses described above led us to assess the potential of p38α and MMP13 as targets for pharmacological intervention. Small-molecule inhibitors of p38α and MMP13, SCIO469 and DB04760, respectively, were tested for their ability to suppress allergic dermatitis. These inhibitors were topically applied to the inflamed skin site concurrently with OVA challenge. Therefore, the inhibitors were administered after sensitivity to OVA had been fully established in the tested animals. The p38α inhibitor-treated skin displayed a significant decrease in all aspects of OVA-induced changes (epidermal and dermal thickening and T cell and eosinophil infiltration; FIGS. 4A-4G). Treatment with the MMP13 inhibitor also significantly reduced OVA-induced epidermal thickening and T cell infiltration (FIG. 4B and FIG. 4E) and resulted in a trend of decrease in edema formation and eosinophil infiltration (FIG. 4C and FIG. 4G; P=0.1888 and P=0.0833, respectively).

These results suggested that blocking p38α or MMP13 activity after allergen sensitization and during active dermatitis could effectively treat allergic disease.

### Example 5: Effects of Genetic Ablation and Pharmacological Inhibition of MMP13 on Allergic Airway Inflammation

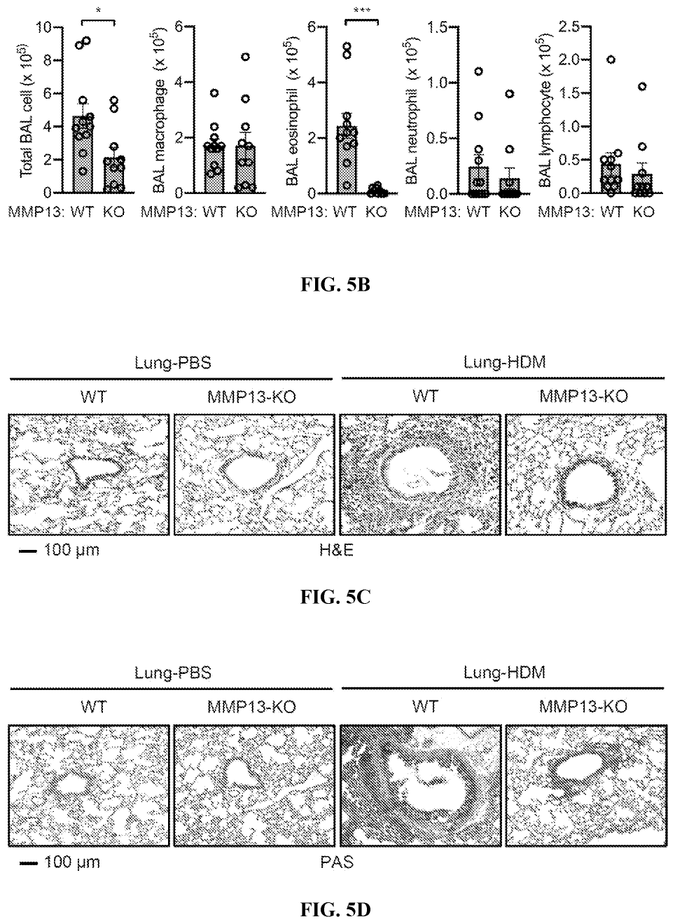

Atopic dermatitis and asthma share key immunologic and pathologic features in their mechanisms for pathogenesis. Whether MMP13 function also contributes to allergic airway inflammation was explored in a mouse model of asthma. This model involved repeated intranasal instillation of the house dust mite (HDM) Dermatophagoidesfarinae. HDM-challenged WT mice developed allergic airway inflammation, as evidenced by an influx of leukocytes into the airway lumen, bronchial wall thickening, and goblet cell hyperplasia, which were detected by the histological analysis of bronchoalveolar lavage (BAL) cells (FIGS. 5A-5B) and lung tissue sections (FIGS. 5C-5D). HDM-challenged MMP13-KO mice exhibited a precipitous decrease in BAL eosinophil numbers while their BAL neutrophils and lymphocyte numbers were comparable to those of WT counterparts (FIG. 5B). MMP13 deficiency also prevented HDM-induced lung interstitial infiltration, bronchial wall thickening and goblet cell hyperplasia (FIGS. 5C-5D).

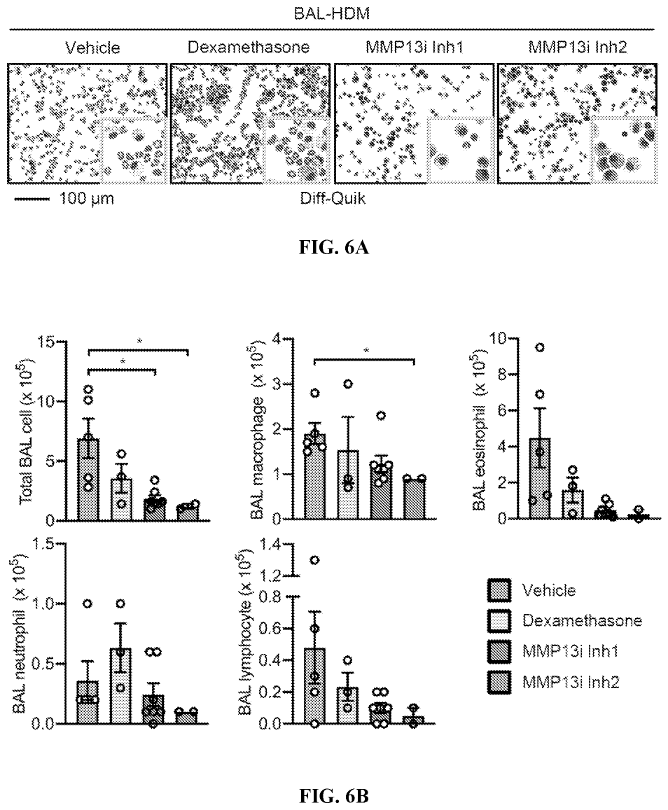

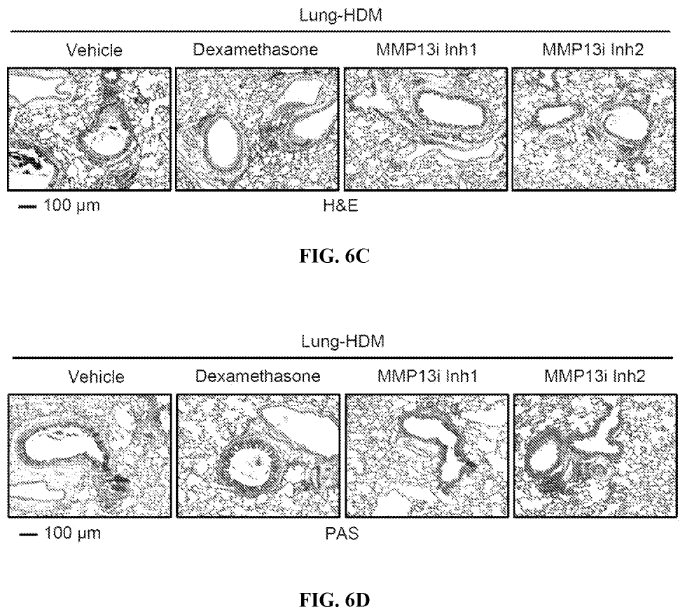

Next, the effects of pharmacological inhibition of MMP13 on HDM-induced allergic airway inflammation was examined. Two small-molecule MMP13 inhibitors, CL82198 and DB04760, were used in this test. These inhibitors, when administered intraperitoneally during active airway inflammation, substantially reduced total BAL leukocyte numbers (FIGS. 6A-6B) and prevented histologically detectable changes in the lung tissue sections (FIGS. 6C-6D). The effectiveness of the two MMP13 inhibitors was superior to that of dexamethasone, which was tested in parallel for comparison.

These results, in conjunction with the findings from inhibitor treatment of mice with allergic dermatitis, showed that pharmacological inhibition of MMP13 was effective even when inhibitor treatment began after the establishment of allergen sensitivity and during the period of active allergic disease.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating an allergic inflammatory disease, the method comprising administering to a subject in need thereof an effective amount of an inhibitor of matrix metalloproteinase 13 (MMP13).

2. The method of claim 1, wherein the inhibitor of MMP13 is selected from the group consisting of a small molecule inhibitor, an anti-MMP13 antibody, a peptide inhibitor, and an agent that inhibits expression of MMP13.

3. The method of claim 2, wherein the inhibitor of MMP13 is a small molecule inhibitor of MMP13.

4. The method of claim 3, wherein the small molecule inhibitor is a non-Zn²⁺-binding MMP13 inhibitor.

5. The method of claim 4, wherein the small molecule inhibitor is selected from the group consisting of CL82198 (CAS 307002-71-7), DB04760 (CAS 544678-85-5), WAY 170523 (CAS 307002-73-9), MMP-9/MMP-13 inhibitor I (CAS 204140-01-2), PD166793 (CAS 199850-67-4), and BI-4394 (CAS 1222173-37-6).

6. The method of claim 5, wherein the small molecule inhibitor is CL82198 (CAS 307002-71-7).

7. The method of claim 5, wherein the small molecule inhibitor is DB04760 (CAS 544678-85-5).

8. The method of claim 1, wherein the inhibitor of MMP13 is formulated for topical administration.

9. The method of claim 1, wherein the inhibitor of MMP13 is formulated for inhalation.

10. The method of claim 1, wherein the inhibitor of MMP13 is formulated in a pharmaceutical composition, which further comprises a pharmaceutically acceptable carrier.

11. The method of claim 1, wherein the subject is a human patient having or at risk for having an allergic inflammatory disease.

12. The method of claim 1, wherein the allergic inflammatory disease is selected from the group consisting of allergic asthma, atopic dermatitis, allergic rhinitis, and allergic conjunctivitis.

13. The method of claim 12, wherein the allergic inflammatory disease is allergic asthma.

14. The method of claim 12, wherein the allergic inflammatory disease is atopic dermatitis.

15. The method of claim 1, wherein the inhibitor of MMP13 is administered topically.

16. The method of claim 1, wherein the inhibitor of MMP13 is administered systemically.

17. The method of claim 16, wherein the inhibitor of MMP13 is administered by inhalation.

18. The method of claim 1, further comprising administering to the subject an additional therapeutic agent.

19. The method of claim 18, wherein the additional therapeutic agent is selected from the group consisting of an anti-inflammatory agent, an antihistamine, an immunosuppressant, an IL-4Rα neutralizing agent, and a JAK inhibitor.

20. The method of claim 18, wherein the inhibitor of MMP13 and the additional therapeutic agent are formulated in a single formulation.
